(19) United States
(12) Patent Application Publication
(10) Pub. No.: US 2021/0299399 A1
(43) Pub. Date: Sep. 30, 2021

(54) ACTIVE / PASSIVE ANTI-PATHOGEN ENDOTRACHEAL TUBE

(71) Applicants: Peter Barrett, Atlanta, GA (US); Joseph J. Bango, New Haven, CT (US); Michael E. Dziekan, Bethany, CT (US)

(72) Inventors: Peter Barrett, Atlanta, GA (US); Joseph J. Bango, New Haven, CT (US); Michael E. Dziekan, Bethany, CT (US)

(21) Appl. No.: 17/218,553

(22) Filed: Mar. 31, 2021

Related U.S. Application Data
(60) Provisional application No. 63/002,904, filed on Mar. 31, 2020.

(51) Int. Cl. A61M 25/00; A61M 16/04; A61L 2/10; A61L 2/03; A61L 2/26

(52) U.S. Cl. CPC A61M 25/00; A61M 16/0434; A61L 2/10; A61M 2025/1061; A61L 2/26; A61M 2025/0019; A61L 2/03

(57) ABSTRACT

An endotracheal tube comprising: a main body; an inflation tube in operational communication with the main body; one or more pairs of silver conductive rings attached to the main body and configured to pass a small current between each pair of conductive rings; and a power supply in communication with the conductive rings.

### CROSS-REFERENCES

This patent application claims the benefit of provisional patent application No. 63/002,904, by Peter Barrett, Joseph J. Bango, and Michael E. Dziekan, entitled “AN ACTIVE/PASSIVE ANTI-PATHOGEN ENDOTRACHEAL TUBE”, filed on Mar. 31, 2020, and which provisional application is fully incorporated by reference herein.

### BACKGROUND

### Field of Invention

The use of endotracheal tubes (ET Tubes) is currently the most rapid and effective means gaining direct access to the human airway. The Endotracheal tube serves as an open passage through the upper airway. The purpose of endotracheal intubation is to permit air to pass freely to and from the lungs in order to ventilate the lungs and can be connected to mechanical ventilator to provide artificial respiration. Artificial or mechanical ventilation is used on a daily bases in the operating room during surgical procedures. Many intensive care unit patients are admitted with pulmonary problems requiring mechanical ventilation. In some cases, the patient is connected to a ventilator for an extended period of time. Due to the excessive moisture content in the breath, moisture and bacteria can build up within the ET tube, which can cause a variety of problems such as pneumonia and ARDS (Acute Respiratory Distress Syndrome). The presence of an endotracheal tube also inhibits the human body's natural mucociliary elevation, and inhibits secretion removal. The development of ventilator associated pneumonia is a common and serious problem in intensive care units. This condition arises when a patient develops a new infiltrate on chest x-ray and has microbiologic positive sputum cultures and a new fever. Ventilator associated pneumonia prolongs the patient's hospital stay, adds to health-care costs and can lead to the development of multi-drug resistant bacteria and increased mortality. In the practice of intensive care medicine various interventions have been bundled together to try to mitigate the development of ventilator associated pneumonia with very limited benefit.

### Description of Prior Art

If the ET tube is inadvertently placed in the esophagus (right behind the trachea), adequate respirations will not occur. Brain damage, cardiac arrest, and death can occur. Aspiration of stomach contents can result in pneumonia chemical injury to the lung and adult respiratory distress syndrome (ARDS). Placement of the tube too deep can result in only one lung being ventilated and can result in a pneumothorax as well as inadequate ventilation. During endotracheal tube placement, damage can also occur to the teeth, the soft tissues in the back of the throat, as well as the vocal cords. It is no wonder that this procedure should be performed by a practitioner with experience in intubation. In the vast majority of cases of intubation, no significant complications occur.

Endotracheal intubation is a procedure by which a tube is inserted through the mouth down into the trachea most frequently under direct vision (the large airway from the mouth to the lungs). Before surgery, this is often done under deep sedation. In emergency situations, the patient is often unconscious at the time of this procedure. A major problem associated with the use of endotracheal tubes is that development of a biofilm or biolayer.

A number of approaches have been developed to address the mucus accumulation problem for endotracheal tubes. In the most basic approach, the mucus laden endotracheal tube is simply removed from the patient's trachea and replaced with a clean endotracheal tube. Needless to say, removing the mucus laden endotracheal tube is very uncomfortable for patient, particularly since ventilation must be interrupted during the removal process. Moreover, reinsertion of a clean endotracheal tube can lead to tracheal injury, particularly if it is done frequently. In addition this practice has been proven not to decrease the development of ventilator associated pneumonia.

In another common approach, normal saline is introduced into the endotracheal tube to dissolve the mucus and a suction catheter is then inserted into the endotracheal tube to try to suction out the dissolved mucus deposits. This suctioning approach has a number of drawbacks. First of all, the suctioning process typically takes 10 to 15 seconds to complete, which can seem like an agonizingly long time for many patients and may lead to hypoxia. Secondly, the suction catheter tends to miss a number of the accumulated mucus deposits and thereby leaves them as a breeding ground for infectious bacteria. Third repeated endotracheal suctioning can lead to localized airway trauma and bleeding.

A further approach to the mucus accumulation problem is described in U.S. Pat. No. 5,687,714. In this approach, droplets of water or saline are entrained in the oxygen/air ventilation mixture to continually dissolve mucus before it has an opportunity to form deposits and a reverse thrust catheter is used to help transport dissolved mucus away from the lungs.

Others have tried to utilize the antibiotic nature of silver to destroy bacterial growth such as the addition of a thin film of silver or silver containing compound. This can be either in a film or colloid form or in the form of a ring or coil. Although these methods have some effectiveness, the effect is short lived due to a thin surface film on the silver.

### SUMMARY OF THE INVENTION

The invention relates to an endotracheal tube comprising: a main body; an inflation tube in operational communication with the main body; one or more pairs of silver conductive rings attached to the main body and configured to pass a small current between each pair of conductive rings; and a power supply in communication with the conductive rings.

### BRIEF DESCRIPTION OF THE DRAWINGS

FIG. 1 shows a diagram of the placement of a commonly used ET tube in the uninfected state. During normal use, the inflatable low-pressure cuff would be fully inflated.

FIG. 2 shows a diagram of a group of small conductive rings located within the inside of the ET tube. The diagram also shows a schematic of an electrical connection to provide electrical stimulus to kill and mitigate any bacterial or viral buildup within the ET tube. The schematic also indicates a means of current limiting to prevent any sparks or harmful shocks.

FIG. 3 shows a diagram of a group of small conductive rings located within the inside of the ET tube. The diagram also shows a schematic of an electrical connection to provide an alternating polarity electrical stimulus to kill and mitigate any bacterial or viral buildup within the ET tube. The schematic also indicates a means of current limiting to prevent any sparks or harmful shocks.

FIG. 4 shows a diagram of a section of another embodiment of the disclosed ET tube.

FIG. 5 shows a diagram of a section of another embodiment of the disclosed ET tube.

### DETAILED DESCRIPTION OF THE INVENTION

The described invention details how to produce an ET tube that will continually mitigate any buildup of bacteria, and help prevent the onset of pneumonia. The importance of proper respiration is understood by even the most naïve person, but what is less understood by the average person are the problems associated with extended usage of an ET tube. As stated earlier, prolonged use of an ET tube can facilitate VAP (Ventilator Associated Pneumonia) and ARDS (Acute Respiratory Distress Syndrome). The described invention details how to produce an ET tube that will continually mitigate any buildup of harmful bacteria or viruses and help prevent the onset of potentially fatal respiratory illnesses.

Silver has been shown to have some remarkable characteristics as a natural biocide. Ancient Greeks and Romans used silver containers to keep liquids fresh. American settlers would often place a silver dollar in milk to delay its souring. Most of the world's airlines use silver filters on aircraft to prevent dysentery. After testing 23 different methods for purifying water, NASA selected silver water filters for use on board the space shuttle. Japanese researchers have found that silver is even able to detoxify some poisons. There is little doubt that silver can be of tremendous use if properly applied to medical devices such as the ET tube. Several researchers have applied silver containing compounds to ET tubes with limited success, but what no one has yet done is to apply both silver and a means for both generating a silver colloid and a small voltage that could destroy harmful pathogens. One embodiment of this invention discloses using an energy source to assist with the silver natural biocide property. If a small current limited electrical stimulus is coupled to two or more silver electrodes attached to the ET tube, then the eventual buildup of any harmful bacteria or viruses can be mitigated. The described invention utilizes a combination of silver or copper (both natural biocides) electrodes in addition to an external electrical stimulus.

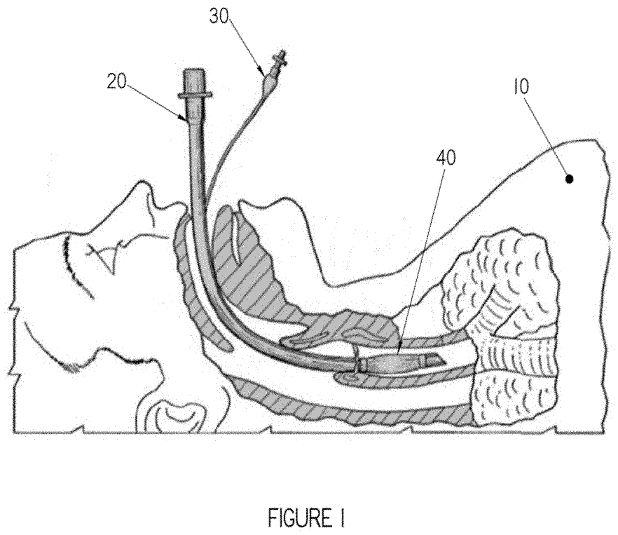

FIG. 1 details how a typical ET tube is used to assist a patient 10 during respiration. The ET tube has three basic parts, the main body of the ET tube 20 that allows air to be passed through to the lungs. Another part is the inflation tube 30 that allows one to inflate the inflatable balloon 40 that will seal the ET tube in place.

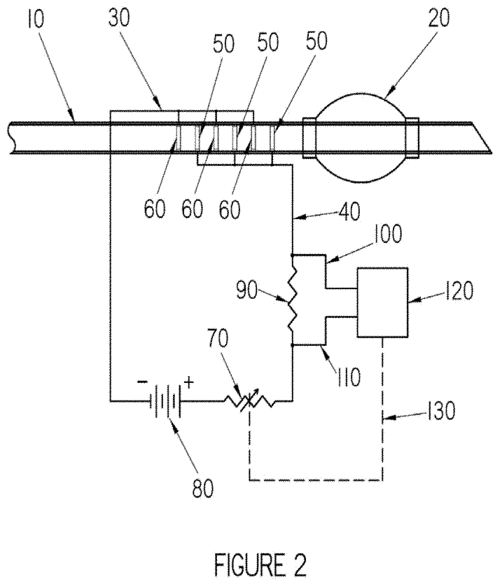

FIG. 2 details a schematic representation of a circuit that will apply power to a set of conductive rings. The main body of the ET tube 10 houses a set of conductive rings 50 and 60 that will be used to pass a small current through each set to conductive rings. The ET tube 10 is shown with the inflatable balloon 20 fully inflated, as would be the case during normal use. The conductive rings 50 and 60 are made of silver, and are positioned a short distance from each other to allow a small current to pass between each pair of closely spaced conductive rings. The current that passes through the conductive rings is brought about by the addition of moisture from the lungs. As a person breathes, the moisture in the lungs can build up inside the ET tube and coat the inside so that the liquid makes contact with the rings, and establishes a current. The amount of current can be adjusted by the control unit 120 and the adjustable current limiting resistor 70. If the current increases beyond a predetermined limit, the control unit 120 will sense this by measuring the voltage across the current sensing resistor 90. The control unit 120 will adjust the current limiting resistor 70 by means of a connection (either mechanical or electrical) 130 to the current limiting resistor 70. If the amount of current going to the conductive silver rings 50 and 60 is too great, the control unit 120 will increase the value of the current limiting resistor 70. A small power source 80 can be either a battery or a wall mounted direct current supply that will provide the needed power. In this operation the voltage maintains a consistent polarity, i.e., there is no polarity change between positive and negative with respect to ground. The voltage is either always positive, or always negative, and does not switch between each. This is known as the direct mode of operation. In this mode a consistent polarity is applied to the conductive silver rings that serve a two-fold purpose. The constant polarity produces a small current that produces a controlled amount of silver colloids within the inside of the ET tube, and also helps to destroy any pathogens in the path of the current between the rings. In addition to the colloidal silver produced by the current flowing through the conductive silver rings 50 and 60, the current also helps to kill the pathogens that might be found inside the ET tube.

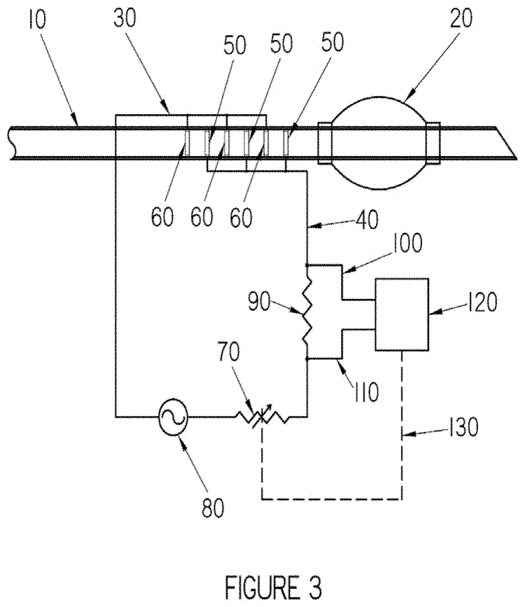

FIG. 3 details a schematic representation of a circuit that will apply power to a set of conductive rings. The main body of the ET tube 10 houses a set of conductive rings 50 and 60 that will be used to pass a small current through each set to conductive rings. The ET tube 10 is shown with the inflatable balloon 20 fully inflated, as would be the case during normal use. The conductive rings 50 and 60 are made of silver, and are positioned a short distance from each other to allow a small current to pass between each pair of closely spaced conductive rings. The current that passes through the conductive rings is brought about by the addition of moisture from the lungs. As a person breathes, the moisture in the lungs can build up inside the ET tube and coat the inside so that the liquid makes contact with the rings, and establishes a current. The amount of current can be adjusted by the control unit 120 and the adjustable current limiting resistor 70. If the current increases beyond a predetermined limit, the control unit 120 will sense this by measuring the voltage across the current sensing resistor 90. The control unit 120 will adjust the current limiting resistor 70 by means of a connection (either mechanical or electrical) 130 to the current limiting resistor 70. If the amount of current going to the conductive silver rings 50 and 60 is too great, the control unit 120 will increase the value of the current limiting resistor 70. A small alternating polarity power source 80 supplies the needed power to produce the required current. In this operation the voltage periodically changes polarity, i.e., there is a polarity change between positive and negative with respect to ground. The voltage is alternating between positive and negative at a predetermined rate with respect to time. This is known as the alternating mode of operation. In this mode a periodically alternating polarity is applied to the conductive silver rings that serve a two-fold purpose. The constant polarity produces a small current that produces a controlled amount of silver colloids within the inside of the ET tube, and also helps to destroy any pathogens in the path of the current between the rings. In addition to the colloidal silver produced by the current flowing through the conductive silver rings 50 and 60, the current also helps to kill the pathogens that might be found inside the ET tube.

It must be emphasized here that although the device can operate in a direct mode or an alternating polarity mode, it can also be operated in a pulsed operation. In the third mode of operation, or pulsed mode, a series of pulses are applied to the sets of conductive ring 50 and 60, where one set is at an opposite or ground polarity with respect to the other set. In the pulsed mode, a constant voltage can be applied to the conductive ring pairs 50 and 60, while the power is interrupted in a repeatable, random, or pseudorandom manner. The polarity can remain the same during pulsed operation, or the polarity can alternate between positive and negative with respect to ground. This does not mean that in pulsed mode that the polarity must alternate between positive and negative polarity, it is an option, and may have several positive pulses applied to the conductive rings 50 and 60, before switching to a negative polarity. It must be understood that a variety of wave shapes could be used for alternating mode of operation. It must also be understood that in the pulsed mode of operation, the polarity may remain constant, with only the current applied to the conductive rings 50 and 60 changes. In this way, the current could be modulated in such a manner as to maintain a consistent polarity, while having a greater or lesser amplitude. In an embodiment of the disclosed invention, the conductive silver rings would also be on the outside of the ET tube to mitigate any buildup of pathogens within the trachea.

### Optical Sterilization

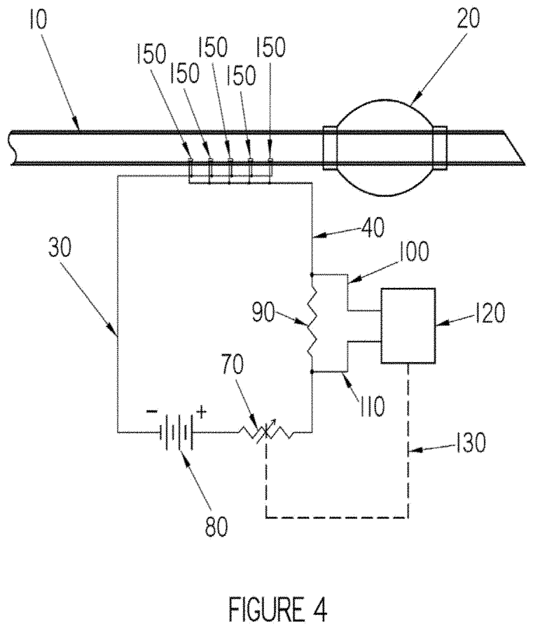

In another embodiment of the invention shown at FIG. 4, ultraviolet light is used to destroy and prevent the growth of any pathogens, either bacteriological or viral, inside the endotracheal tube surface. Ultraviolet light is defined as being (of electromagnetic radiation) having a wavelength shorter than that of the violet end of the visible spectrum but longer than that of X-rays. The preferred light is beyond the violet in the spectrum, corresponding to light having wavelengths shorter than 4000-angstrom units. The wavelength of UV radiation ranges from 328 nm to 210 nm (3280 A to 2100 A). Its maximum bactericidal effect occurs at 240-280 nm. Mercury vapor lamps emit more than 90% of their radiation at 253.7 nm, which is near the maximum microbicidal activity 775. Inactivation of microorganisms results from destruction of nucleic acid through induction of thymine dimers. Ultraviolet light dispersed inside the endotracheal tube also prevents and pathogens from forming or living in a biofilm that may form on the inside wall of the tube.

In an embodiment, the inside wall of the tube is coated with a vapor deposition of aluminum or other non-toxic metal to stop ultraviolet rays from penetrating the tube walls and encountering external tissue. In this process, aluminum, gold, or other suitable metal is evaporated in a vacuum chamber. The vapor then condenses onto the surface of the polymer tube substrate, leaving a thin layer of metal coating. The entire process takes place within a vacuum chamber to prevent oxidation. This deposition process is also commonly called physical vapor deposition.

In another embodiment, the tube may be constructed of a polymer that normally impedes the passage of ultraviolet light. Since long-term UV light may cause many polymers to degrade over time, a UV blocking polymer may be used for an endotracheal tube. In one adaptation of the disclosed invention, a highly ductile UV-shielding polymer may be created using boron nitride additives. Polymer composites with enhanced mechanical, thermal or optical performance usually suffer from poor ductility induced by confined mobility of polymer chains. Herein, highly ductile UV-shielding polymer composites are successfully fabricated. Boron nitride (BN) materials, with a wide band gap of around ˜6.0 eV, are used as fillers to achieve the remarkably improved UV-shielding performance of a polymer matrix. In addition, it is found that spherical morphology BN as a filler can keep the excellent ductility of the composites. For a comparison, it is demonstrated that traditional fillers, including conventional BN powders can achieve the similar UV-shielding performance but dramatically decrease the composite ductility. The mechanism behind this phenomenon is believed to be lubricant effects of BN nanospheres for sliding of polymer chains, which is in consistent with the thermal analyses. This study provides a new design to fabricate UV-shielding composite films with well-preserved ductility.

The source of the ultraviolet light can be derived from a single light emitting diode (LED) or a string of UV LEDs affixed onto the inner surface of the Endotracheal tube. However, to reduce manufacturing costs, in an embodiment, the ultraviolet light source is external to the endotracheal tube and supplied to the tube by an external attachment that function as a light pipe or fiber optic pathway. The light is conducted into the tube and is dispersed within the tube to kill any possible pathogens. The aforementioned metalization and/or UV opaque polymer material keeps the UV light confined within the tube proper.

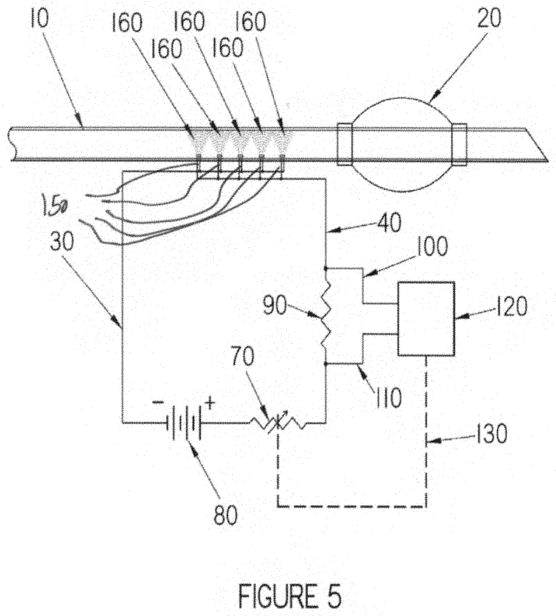

Referring to FIG. 5, in an embodiment, power provided externally by a battery, DC or AC power source 80 is current regulated by a constant current source 120, with the current being measured indirectly as a voltage across resistor 90 and connected to the constant current source 120 by leads 100 and 110 respectively. The power to the ultra violet LED or LED array is connected to the ultraviolet light source or sources by leads 30 and 40 respectively. Dispersed ultraviolet light 160 destroys and any pathogen along the inner surface of the tube and precludes the development of a biofilm.

It would be obvious to those skilled in the art that a variation of the arrangement of ultraviolet light sources either within or external to the endotracheal tube may be created. In an embodiment, the ultraviolet light sources are distributed along the inner surface of the tube, are surface mount LED's, and distributed in a spiral so as to cover the entire inner tube surface 360 along the entire length of the tube. In an embodiment, the ultraviolet light will be pulsed to reduce thermal losses and to reduce power consumption. The tube will be preferably made of a UV absorbing polymer so as to mitigate UV light passage through the tube and/or be metallized or covered with a UV absorbing or reflecting material. In a final embodiment, the light source may be located external to the tube, conducted within the tube using a fiber optic or other light pipe non-attenuating to ultraviolet light, and which disperses said light within the tube to sterilize the walls and preclude pathogen growth.

### REFERENCE NUMERALS

FIG. 1:
10 Diagram of a section of the human body detailing the head and upper portion of the thoracic cavity.
20 End section of the ET tube that would be connected to a ventilator or ventilation means.
30 End section of the ET tube that would be connected to an air hose that would manually inflate the inflatable ball to form a tight seal when in the trachea.
40 Inflatable section of the ET tube that would be manually inflated to form a tight seal when in the trachea.

FIG. 2:
10 Diagram of a section of an ET tube.
20 Inflatable section of the ET tube to form seal within trachea.
30 Schematic of connection between power source and one or more conductive rings within the ET tube.
40 Schematic of connection between power source and one or more conductive rings within the ET tube.
50 Diagram of conductive rings within the ET tube.
60 Diagram of conductive rings within the ET tube.
70 Schematic drawing of a variable resistance used to limit and control the current applied to the conductive rings.
80 Schematic diagram of a DC power source that will be used to provide power to the conductive rings within the ET tube.
90 Schematic drawing of a resistor that will be used to measure the amount of current flowing to the conductive rings.
100 Schematic of connection between one side of the current monitoring resistor and the input to the control unit.
110 Schematic of connection between the opposite side of the current monitoring resistor and the input to the control unit.
120 Diagram of a box that represents the control unit that will monitor the current flowing through the current monitoring resistor and will make adjustment to the current limiting resistor to prevent any danger of sparking or shock.
130 Schematic diagram showing a link between the control unit and the current limiting resistor that allows for the current limiting resistor to be adjusted by the control unit.

FIG. 3:
10 Diagram of a section of an ET tube.
20 Inflatable section of the ET tube to form seal within trachea.
30 Schematic of connection between power source and one or more conductive rings within the ET tube.
40 Schematic of connection between power source and one or more conductive rings within the ET tube.
50 Diagram of conductive rings within the ET tube.
60 Diagram of conductive rings within the ET tube.
70 Schematic drawing of a variable resistance used to limit and control the current applied to the conductive rings.
80 Schematic diagram of an alternating polarity power source that will be used to provide power to the conductive rings within the ET tube.
90 Schematic drawing of a resistor that will be used to measure the amount of current flowing to the conductive rings.
100 Schematic of connection between one side of the current monitoring resistor and the input to the control unit.
110 Schematic of connection between the opposite side of the current monitoring resistor and the input to the control unit.
120 Diagram of a box that represents the control unit that will monitor the current flowing through the current monitoring resistor and will make adjustment to the current limiting resistor to prevent any danger of sparking or shock.
130 Schematic diagram showing a link between the control unit and the current limiting resistor that allows for the current limiting resistor to be adjusted by the control unit.

FIG. 4:
10 Diagram of a section of an ET tube.
20 Inflatable section of the ET tube to form seal within trachea.
30 Schematic of connection between power source and one or more ultraviolet light emitting diodes within the ET tube.
150 Ultraviolet light emitting diodes positioned along the wall of the tube

FIG. 5:
10 Diagram of a section of an ET tube.
20 Inflatable section of the ET tube to form seal within trachea.
30 Schematic of connection between power source and one or more ultraviolet light emitting diodes within the ET tube.
160 Ultraviolet light being dispersed within an endotracheal tube by a single or series of light emitting diodes (LED's) positioned along the wall of the tube.

### Anti-Biofilm Surface Coating

In yet embodiment of the proposed invention, specialized surface treatments of the endotreacheal tube polymer inner and outer surface can reduce or eliminate most infectious supporting biofilm production, and, in combination with suitable silver bearing additives articulated previously in this patent disclosure, and/or in combination with suitable ultraviolet irradiation, can substantially reduce or eliminate endotreaceal tube induced infections in patients.

Nonspecific protein adsorption on material surfaces, which is caused by surface energy, intermolecular forces, hydrophobicity, and ionic or electrostatic interaction, is a major problem for implanted materials because it can initiate blood coagulation and thrombus formation as well as complement activation and lead to an inflammatory response within the surrounding tissue. It also hinders the effectiveness of biosensors or drug delivery vehicles that are supposed to interact with the body, and which can foster infection in an endotreacheal tube.

Therefore, many bioinert and bioactive materials have been developed to improve the antifouling ability of biofluid contacting materials, including surface coatings consisting of poly(ethylene glycol) (PEG) or oligo(ethylene glycol) (OEG), polyvinylpyrrolidone, peptoids, zwitterionic polymers, and polysaccharides. PEG, which is a linear, flexible, hydrophilic, and water-soluble polyether, is most frequently used in such types of surface modifications.

In the search for antifouling PEG alternatives, polyglycerol (PG) and its derivatives have been identified as strong and potent candidates because of their easy accessibility and higher thermal and oxidative stability than that of PEG. Hyperbranched polyglycerol (hPG), which has a highly branched architecture consisting of a flexible aliphatic polyether backbone with hydrophilic surface groups, shows similar or better protein-resistant and less thrombocyte-activating performance than PEG. Gold, glass, and poly(ether imide) surfaces have been modified by hPG monolayers and were classified as highly protein-resistant materials.

However, it still remains a challenge to immobilize hPG on a broad range of different material surfaces, like titanium oxide and many plastics, by using a universal surface linker group because of the relative chemical inertness of those materials. In one paper, Multivalent Anchoring and Cross-Linking of Mussel Inspired Antifouling Surface Coatings Biomacromolecules, June 2014, by Tobias Becherer et al, a new method using Catechol was revealed to mitigate biofilm production by making the surface more hydrophobic through a reduction in surface energy.

Catechol has been proven to be a powerful anchor for surface modification. Catechol groups, which are found in mussel adhesive proteins and bacterial siderophores, can adhere on virtually almost any material surface. Although the mechanistic adhesion details are still not well understood, previous studies have proposed several mechanisms for different kinds of substrates. Some research has proposed that a charge-transfer complex could be formed between the catechol and TiO2 surface or that a hydrogen bond could be formed between the catechol and mica surface. Also van der Waals' force between the catechol and inert polymer surfaces and covalent bonds on nucleophile-containing surfaces have been discussed. Multiple catechol units are required in the anchor group to effectively and stably immobilize macromolecules on substrates. Thus, a number of new catecholic anchor groups have been developed, including 3,4-dihydroxyphenylalanine (DOPA) short peptides, pentapeptide of alternating DOPA and lysine residues, catechol side chains, oligo-catechol, tripodal catecholates, and polyDOPA. It is not easy to prepare these multiple catecholic anchor groups, however, because they require challenging organic synthesis or solid phase synthesis for the DOPA-containing peptides. So far, most of the catechol-bearing anchor groups, however, have been synthesized on a milligram scale only, which is insufficient for many coating applications. Moreover, the catecholic side chain grafted linear polymers may coil to inhibit the effectiveness of the catechols. In addition, in many cases a second coupling step of the bioinert or bioactive compound or polymer to the catecholic anchor moiety is required. Although catechols had been employed as the terminal groups of multiarmed PEG derivates to prepare geltype coatings, the limited number of arms (normally only 4-8) limited the amount of grafted catechols and the free chains.

Catechol-functionalized hPGs with a degree of functionalization (DF) of statistic 1 equiv, 5%, 10%, and 30% were developed to investigate the effects of the catechol amount on the stability and antifouling performance of the coatings (as shown in Table 1). A DF of 30% is already near the maximum grafting ratio of catechols on hPG (max ca. 40%). With a DF of 30%, the water solubility decreased dramatically so that hPGCat30 is only soluble in strong polar organic solvents like dimethylformamide (DMF). In addition to catechols, amine groups are also helpful to enhance the adherence of the mussel adhesive proteins. Amines can couple with catechols under oxidizing conditions via Michael addition or Schiff base reactions. In this regard, free amines containing catecholic hPGs with different catechol/amine molar ratios were also synthesized.

Surface Characterization. Because of their highly branched architecture and large number of functional groups on the surface of hPG, catecholic hPGs can be cross-linked with each other by oxidation of the catechol groups to enhance the stability of the coatings. Thus, catecholic hPGs can be used to modify many different substrates including ceramic and polymer surfaces, while one side catechol-functionalized macromolecules, i.e., terminal functionalized linear polymers or focal point functionalized dendrons, have been reported to adhere only to metal and metal oxide surfaces. Within the present study, a large variety of different substrates, including TiO2 (metal oxide), gold (noble metal), aluminum (metal with native oxide surface), SiO2 (nonmetal oxide), glass (ceramic), polystyrene (PS, polymer), as well as polypropylene (PP, polymer) have been successfully treated with catecholic hPGs.

UV-vis analysis was used to detect the oxidation and crosslinking status of the catechols in different buffers. The absorption peaked around 350 nm because of quinone formation. 52 The MOPS buffer at pH 7.5 is alkaline enough to oxidize some of the catechol groups and cause cross-linking. In PB buffer at pH 8.5, more catechol groups could be oxidized to quinone groups, which are advantageous for coating the chemical inert polymer surfaces, like PS and PP.

Theoretically, there are two possible ways to form coatings on substrates (Figure S2, Supporting Information). First, some catecholic hPG molecules directly adsorb onto the substrate surface; then, other molecules can become immobilized by cross-linking catechols with surface bonded molecules, which

imitates a “grafting from” approach. Second, catecholic hPGs can cross-link with each other to form aggregates in solution, which is followed by adsorption of these aggregates onto the substrates as “grafting to”. The average size of the hPG-Cat10 molecules in pH 7.5 MOPS buffer was 4.8 nm in a freshly prepared solution and increased to 6.6 nm after 8 h of standing at room temperature (measured by dynamic light scattering). This indicates partial cross-linking of the hPG-Cat10 molecules in solution. Static water contact angle was introduced to evaluate the effect of the catecholic hPG coating on the substrates. After modification, the angles on all of the substrates changed dramatically to be close to 20°, which was the typical static water contact angle of the hPG-monolayer-coated gold surfaces. The water contact angles of modified TiO2, gold, aluminum, SiO2, and glass are slightly larger than 20°, due to the free catechols on the surface. Only the angles of coated PS and PP are a little higher at about 30° and 36°, respectively. This effect can be explained by the weaker interaction between catechol groups and inert polymer surfaces, i.e., only van der Waals' forces. In general, the hPG-Cat10 coatings were successfully immobilized on all substrates. The surface energy of TiO2 surfaces coated with different catecholic hPG samples with varying amounts of catecholic groups was calculated from the advancing contact angles of three kinds of liquid with different polarities: water, diiodomethane, and ethylene glycol. The surface energies of these catecholic hPG-coated TiO2 surfaces were almost the same, approximately 60 mN/m, except for hPG-Cat30. The observed values were obviously higher than the value of bare TiO2: 50.16 mN/m. This demonstrates that all of the used catecholic hPGs can cover the TiO2 surface effectively. The surface energy of hPG-Cat30-coated TiO2 was even higher (65.56 mN/m); and its advancing ethylene glycol contact angle decreased to zero. In this case, the droplet of the test liquid was most likely sucked by the framework of the hPG-Cat30 coating. Exclusively amine functionalized hPG (hPG-A10) also increased the surface energy of the TiO2 (56.52 mN/m), but the value was much lower than that for all of the catecholic hPGs.

TABLE 1
Synthesized Functional Hyperbranched Polyglycerols (hPG)

| Abbreviation | Composition |
|---|---|
| hPG-Cat1 | 1 equiv of catechol per hPG |
| hPG-Cat5 | 5% (4-5 equiv) of catechols per hPG |
| hPG-Cat10 | 10% (8-10 equiv) of catechols per hPG |
| hPG-Cat30 | 30% (12-15 equiv) of catechols per hPG |
| hPG-Cat3.5-A6.5 | 3.5% of catechols and 6.5% of amines per hPG |
| hPG-Cat6.5-A3.5 | 6.5% of catechols and 3.5% of amines per hPG |
| hPG-A10 | 10% (12-15 equiv) of amines per hPG |
| hPG-ester-Cat5 | 5% of catechols with ester bonds per hPG |
| hPG-ester-Cat10 | 10% of catechols with ester bonds per hPG |

### Cell Experiments

An hPG-Cat10 half-coated TiO2 slide was incubated in L929 fibrosarcoma cells in medium for 3 days. The results clearly demonstrates that the hPG-Cat10 coating prevents cell adhesion of L929 adherent cells, as only a few single cells were found on the coated part of the slide, while common cell adhesion was observed on the uncoated part after 3 days of cell exposure. The cells near the border of the uncoated part were confluent, but they still could not grow over the boundary. In order to evaluate the cell resistance of different hPG-Catmodified TiO2 surfaces, eight kinds of slides (bare TiO2, hPGA10, hPG-Cat1, hPG-Cat5, hPG-Cat10, hPG-Cat30, hPGester-Cat5, and hPG-ester Cat10-coated slides) were exposed to NIH3T3 fibroblast cells for 3 days, 7 days, and 14 days. After 3 days of incubation, the surface of bare TiO2 was almost full of cells, and all of the cells were well spread. Some cells adhere to one another to form colonies on the hPG-A10-, hPG-Cat1-, and hPG-Cat30-modified surfaces, but only a few or no cells were on the surface of the hPG-ester-Cat5-, hPG-ester-Cat10-, hPG-Cat5-, and hPG-Cat10-coated slides. After 7 days the surface of bare TiO2 was completely covered, and the cell amount was significantly increased on the hPG-A10-, hPG-Cat1-, and hPG-Cat30-modified surfaces. The extent of single cell spreading increased on the hPG-A10 and hPG-Cat1 surfaces because of the higher density of the binding sites on the unstable coatings. There were still only a few or no cells on the surface of hPG-ester-Cat5-, hPG-ester-Cat10-, hPG-Cat5-, and hPG-Cat10-coated slides. After 14 days of incubation (FIG. 7), the surface of bare TiO2 was very confluent, and the mean cell number reached 1542±368 cells/mm2. The cells had grown over most parts of the hPG-A10- and hPG-Cat30 modified surfaces reaching the number of 270±108 and 530±114 cells/mm2 for each and spreading flat. The number of cells on the hPG-Cat1 surface was smaller: 136±42 cells/mm2. The previous spread of single cells adhere to each other to increase both number and size of the colonies. Meanwhile, fewer cells could be found on the hPG-ester-Cat5 and hPG-ester-Cat10 surfaces, 22±8 and 10±8 cells/mm2, respectively. However, single cells started to spread probably due to the degradation of the ester bonded coatings. The best antifouling results were from the hPG-Cat5 and hPG-Cat10 surfaces because very few cells could adhere on the surfaces and still keep their spherical shape. Besides the TiO2 surface, hPGCat10 also prevented cell adhesion on gold, glass, and PS surfaces. Sarcoma osteogenic (Saos-2) cells were also used to test the above coatings and showed results similar to those of NIH3T3 cells. The cell amount on the surface agrees with the protein adsorption results shown above. Cell adhesion is mediated by the adsorbed protein layer on the surface. Therefore, protein resistant hPG-Cat10 and hPG-Cat5 coatings also effectively resist cell adhesion. hPG-A10 and hPG-Cat1 are not stable enough on the surfaces to prevent cell adhesion over a long incubation time. These results are also in accordance with the stability tests that were performed with fibrinogen and analyzed by QCM. hPG-Cat30 contains too many catechol groups which are able to capture proteins from the cell medium and lead to cell adhesion. Although the cell-resistant performance of the ester-bonded coatings (hPG-ester-Cat5 and hPG-ester-Cat10) is less stable in long-term testing as compared to that of the amide-bonded coatings (hPG-Cat5 and hPG-Cat10), due to the slow hydrolysis of the ester bond, it is worth using the ester bonded coatings in the short-term due to their easy and well established synthesis process. Since the hPG-Cat10-coated PS surface adsorbed a larger amount of proteins than other modified surfaces, the largest amount of cells adhered to it. In addition, cytotoxicity tests (WST-1) did not show any toxicity for hPG-Cat1 and hPG-Cat10. the catecholic hPGs also exhibited similar good tolerability as unmodified hPG49 or cross-linked catechols.

It has therefore been shown that mussel-inspired catechol-functionalized hyperbranched polyglycerol (hPG) does improve the antifouling performance of various surfaces. The results reveal that multivalent catechol anchoring and cross-linking are important for sufficient surface coverage, but too many free catechols lead to protein adsorption and cell adhesion. Five to 10 percent catechols per hPG showed the best stability and antifouling performance. They contain enough catechol groups as anchors and crosslinkers and, at the same time, only expose a few free catechols on the surface. Thirty percent catechols switch the bioinert hPG to a protein-adhesive compound, but a single catechol is not strong enough to stably tether hPG on the surface. Crosslinking under slightly basic conditions has been shown to lead to the formation of a multilayer and enhance the stability of coatings. Compared with the monolayers of catecholterminated molecules that have not been cross-linked, the multilayer coatings of our catecholic hPGs show better stability. Besides the remarkable antifouling performance, catecholic hPGs also show very low cell toxicity, which is relevant for temporary implant materials. Therefore, new multivalent hPG derivatives can be considered as potent alternatives to PEG for a broad range of material surface applications. Additionally, multiple free hydroxyl groups of catecholic hPGs can be further functionalized for many purposes. This catecholic hPG can also work as a macro-anchor to immobilize different functional molecules on surfaces to combine bioinertness and biospecificity.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. An endotracheal tube comprising:
a main body;
an inflation tube in operational communication with the main body;
one or more pairs of silver conductive rings attached to the main body and configured to pass a small current between each pair of conductive rings; and
a power supply in communication with the conductive rings.

2. The endotracheal tube of claim 1, further comprising:
a circuit in operational communication with the one or more pairs of silver conductive rings, the circuit comprising:
a power source; and
a control unit in signal communication with the power source, and where the control unit is configured to adjust the current supplied to the one or more pairs of silver conductive rings.

3. The endotracheal tube of claim 2, wherein the power source is a direct current power source.

4. The endotracheal tube of claim 2, wherein the power source is an alternative current power source.

5. The endotracheal tube of claim 2, wherein a small current supplied to the one or more pairs of silver conductive rings produces silver colloids inside of the main body which can kill pathogens inside the main body, and wherein the current flowing between the pairs of silver conducting rings kills pathogens in the path of the current between the silver conducting rings.

6. The endotracheal tube of claim 2, wherein the control unit provides a pulsed current to the one or more pairs of silver conductive rings.

7. The endotracheal tube of claim 6, wherein the one or more pairs of sliver conductive rings are located on the outside of the main body to mitigate build of pathogens within the trachea.

8. The endotracheal tube of claim 1, further comprising a coating on the inside wall of the inflation tube and main body, the coating configured to prevent ultraviolet rays used to sterilize the inflation tube and main body from penetrating the inflation tube and main body walls and prevent the ultraviolet rays from harming the tissue of the person being intubated.

9. The endotracheal tube of claim 8, wherein the coating is a vapor deposition of aluminum or other non-toxic metal.

10. The endotracheal tube of claim 9, where a coating application to the inside wall of the inflation tube and main body comprises the steps of evaporating non-toxic metal in a vacuum chamber, allowing a metal vapor to condense onto the inside wall of the inflation tube and main body leaving a thin layer of metal coating, and preventing oxidation by performing this coating application in a vacuum chamber.

11. The endotracheal tube of claim 1, where the main body comprises a UV blocking polymer.

12. The endotracheal tube of claim 11, where the UV blocking polymer has fillers of boron nitride materials with a wide band gap of about 6.0 eV.

13. The endotracheal tube of claim 1 further comprising an anti-biofilm coating on the inner and outer surfaces of the main body and the inflation tube.

14. The endotracheal tube of claim 13, wherein the anti-biofilm coating comprises catechol.

15. The endotracheal tube of claim 1, further comprising:
a constant current source in operational communication with the power supply, a resistor attached in parallel to the constant current source, with the circuit configured to allow current being measured indirectly as voltage across the resistor; and
one or more ultraviolet light sources attached in series with the resistor, k the ultraviolet light sources configured to direct ultraviolet light to the main body, and the ultraviolet light sources configured to destroy pathogens on the inner surface of the main body and prevent the development of a biofilm.
